(19) United States
(12) Patent Application Publication
(10) Pub. No.: US 2026/0038643 A1
(43) Pub. Date: Feb. 5, 2026

(54) SYSTEMS AND METHODS FOR MATERIAL CLASSIFICATION

(71) Applicant: Rensselaer Polytechnic Institute, Troy, NY (US)

(72) Inventors: Matthew A. Oehlschlaeger, Cliffton Park, NY (US); M. Arshad Zahangir Chowdhury, Troy, NY (US)

(73) Assignee: Rensselaer Polytechnic Institute, Troy, NY (US)

(21) Appl. No.: 18/667,402

(22) Filed: May 17, 2024

Related U.S. Application Data
(60) Provisional application No. 63/467,753, filed on May 19, 2023.
(60) Provisional application No. 63/648,373, filed on May 16, 2024.

(51) Int. Cl. G16C 20/20; G16C 20/70

(52) U.S. Cl. CPC G16C 20/20; G16C 20/70

(57) ABSTRACT

The systems and methods classify material samples, particularly gas mixtures, by receiving spectrum signal data related to a spectrum of a sample, converting the signal data into a set of spectrum values, reducing, in a feature extraction block with at least one convolutional layer and at least one pooling layer, the set of spectrum values to a set of derived values each indicative of a spectral feature of the spectrum of the sample; and classifying, in a classification block with at least one dense layer and an output layer, the set of derived values as indicative of one or more materials in the sample.

### CROSS-REFERENCE TO RELATED APPLICATIONS

This application claims the benefit of U.S. Provisional Patent Application No. 63/467,753, filed May 19, 2023, and U.S. Provisional Patent Application No. 63/648,373 filed May 16, 2024, which are incorporated by reference as if disclosed herein in their entireties.

### BACKGROUND

Gas sensing in industrial, scientific, and environmental applications, such as process control and remote monitoring, often requires the determination of the speciation of complex mixtures from spectra containing dozens to perhaps hundreds of features, obtained using spectroscopy or other methods. Such gas-sensing applications include the continuous sensing of targeted toxic chemical species as required in personnel safety scenarios, occupational health, pharmaceutical and medical, automotive, national security, pollutant monitoring, and other safety applications. Complex spectra, arising from multicomponent mixtures, can be analyzed using multiple component analysis, independent component analysis, multi-variate calibration, self-modeling curve resolution, and multivariate curve resolution; however, these methods typically require knowledge of the species present in the mixture prior to their application.

Furthermore, to determine quantitative concentrations of mixture components, a regression problem needs to be solved, in which a signal or feature is fit against a calibration or model that defines the concentration.

One-dimensional convolutional neural networks (CNNs) have been used to extract nonlinear features to classify pure gases in a solid-state electronic nose sensor. Classical machine learning models, for instance, classification trees, random forests, multilayer perceptrons, and support vector machines, have been demonstrated to achieve high classification accuracy for the identification of pure gases from terahertz (THz) and infrared (IR) spectra.

What is desired are systems, methods, and devices for material classification, including from multi-component mixtures, and particularly in the field of gas sensing.

### SUMMARY

Some embodiments of the present technology are directed to systems for classifying material samples, including gas mixtures comprising multiple gaseous components. Some embodiments include a one-dimensional convolutional deep learning neural network that comprises a feature extraction block comprising convolutional and pooling layers and a classification block comprising one or more dense layers and an output layer. In some embodiments, the system input is spectrum data of a material sample converted into a set of spectrum values representing the spectral features of the sample. In some embodiments, the spectrum is an absorbance spectrum. In some embodiments, the output layer comprises a neuron for each material or mixture class on which the model has been trained.

Other embodiments of the present technology are directed to methods for classifying material samples that utilize a one-dimensional convolutional deep learning neural network to filter and extract relevant features from the spectrum of a sample, and to classify the sample based on the extracted features. Other embodiments include software products, stored on non-transitory computer-readable media, for performing the operations and methods described herein.

Various embodiments of the technology will now be described with reference to the drawings.

### BRIEF DESCRIPTION OF THE DRAWINGS

The drawings show embodiments of the disclosed subject matter for the purpose of illustrating the technology. However, it should be understood that the present application is not limited to the precise arrangements and instrumentalities shown in the drawings, wherein:

FIG. 1 shows a schematic view of a system according to one embodiment of the present technology.

FIG. 2a-d show simulated reference absorbance spectra for pure gases, where were used to generate simulated mixture spectra for training the convolutional neural network model in the embodiment of FIG. 1.

FIG. 3 shows classification performance on five experimental mixture spectra by the convolutional neural network model in the embodiment of FIG. 1.

FIG. 4 shows classification performance on five experimental single-component spectra by the convolutional neural network model in the embodiment of FIG. 1.

FIG. 5 shows class activation maps (Grad-CAM heat maps) for the classification of three simulated validation spectra by the convolutional neural network model in the embodiment of FIG. 1.

FIG. 6 shows spectra, Grad-CAM heat maps, and the corresponding model mixture prediction softmax scores against experimental data for a model according to a second embodiment.

### DETAILED DESCRIPTION

One or more embodiments of the present technology are directed to a system for material classification, and in particular, for doing so by classifying spectra related to a material sample. In some embodiments, the spectra are absorbance spectra. In some embodiments, the absorbance spectra are generated in the THz frequency region (0.1-10 THz). In some embodiments, the absorbance spectra are generated in the IR frequency range (400 THz to 300 GHz). Other frequency ranges are used in other embodiments. In some embodiments, the spectra are absorption spectra. In some embodiments, the spectra are transmission spectra.

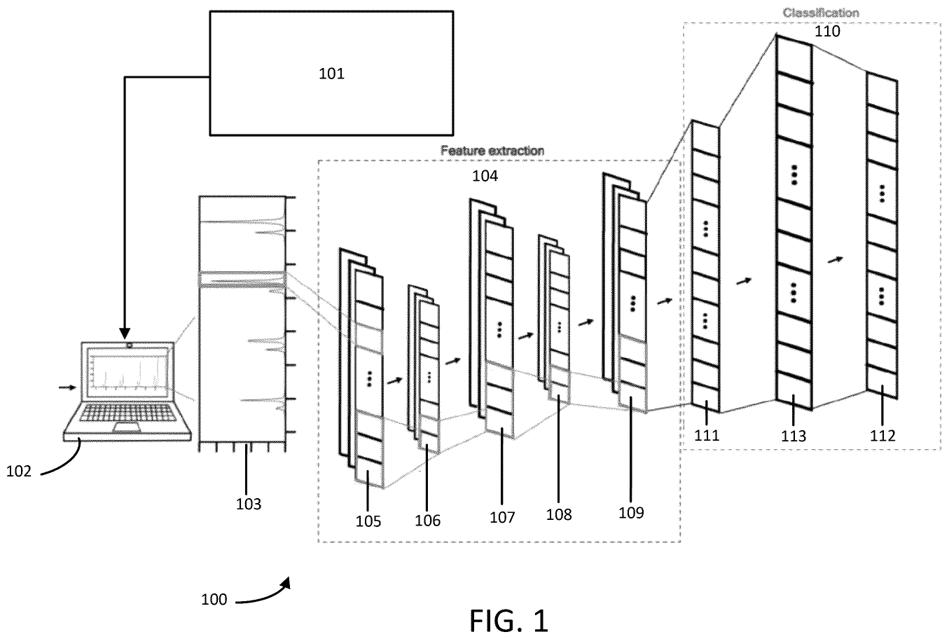

A first embodiment of a system 100 for classifying spectra is shown schematically in FIG. 1. In some embodiments, the system 100 includes a source 101 of spectrum signal data related to a spectrum of a sample. In some embodiments, the source includes a gas cell into which one or more gas samples can be pumped and a source of radiation to be directed through the sample in the gas cell. In some embodiments, the source of radiation is a frequency multiplied THZ or IR source. The source produces spectrum signal data related to a spectrum of a sample, such as a gas or mixture of gases.

In the embodiment shown in FIG. 1, the system 100 further comprises a computer 102. In this embodiment, the computer includes a processor and a computer-readable storage medium. In other embodiments, the system may be configured to access remote processor(s) and/or storage medium components. In some embodiments, the processor includes a plurality of processing units, which includes, but is not limited to, general-purpose processing units, graphical processing units, parallel processing units, etc. In some embodiments, the computer-readable storage medium includes one or more of the following types of memory: semiconductor firmware memory, programmable memory, non-volatile memory, read only memory, electrically programmable memory, random access memory, flash memory, magnetic disk memory, and/or optical disk memory. Either additionally or alternatively system memory may include other and/or later-developed types of computer-readable memory.

The computer-readable storage medium in the system 100 includes instructions executable by the processor. These instructions include receiving spectrum signal data related to a spectrum of a sample and converting the signal data into a set of spectrum values 103. In some embodiments, this includes converting an absorption spectrum into a set of discrete absorbance values. The number of absorbance values in the set is determined based on the desired resolution for the data. In the embodiment of FIG. 1, 229 absorbance values are extracted from a frequency range of 220 to 330 GHz in each spectrum, giving a frequency resolution of 0.016 cm⁻¹. Other embodiments use different resolutions and different wavenumber ranges. In the embodiment shown in FIG. 1, the set of spectrum values comprise an input vector having size (229,1).

The system further comprises instructions for reducing, in a feature extraction block 104 comprising at least one convolutional layer 105 and at least one pooling layer 106, the set of spectrum values to a set of derived values each of which are indicative of a spectral feature of the spectrum of the sample. The feature extraction block uses convolutional and pooling operations to extract features from the input spectrum signal data in the form of the set of spectrum values. These operations reduce the high-dimensional input to the network by capturing the component-distinctive spectral features. In some embodiments, a single convolutional layer and pooling layer pair is used in the feature extraction block. In some embodiments, repeated convolution and pooling operations are used. In some embodiments, the feature extraction block comprises a first convolutional layer, a second convolutional layer, and a first pooling layer between the first and second convolutional layers. These combined processes filter and downsample their inputs to extract the strongest “fingerprinting” features; i.e., they remove information that is less important and extract the information that is more important for eventual identification of the spectrum. Each combined convolutional/pooling layer has adjustable parameters (kernel, padding, stride) which determine how much filtering/downsampling is performed.

The convolution operation involves traversing a sliding window, which is a randomly initialized filter kernel, over the input to the convolutional layer, which captures important spectral feature information while marginally reducing the dimensionality. In the case of the convolutional layer 105, the input is the set of spectrum values extracted from the raw spectrum signal data.

The terms “feature extraction block” are used for ease of understanding some of the functions of the technology, and are not intended to limit the structure or grouping of software code or modules used in embodiments of the present technology.

Each convolution converts the input vector to a new vector whose size is given by (1/S)(W−Kₛ+2P)+1, where W, Kₛ, P, and S are the size of the input, kernel, padding, and the stride, respectively. In some embodiments, three filters were used and provided good classification performance and did not substantially reduce the input dimensionality in each layer. In the embodiment shown in FIG. 1 in which the input vector has size (229, 1), the output of the first convolutional layer is 227×3. In some embodiments, the convolutional layer applies three filters with a kernel size of three. In some embodiments, the convolutional layers, such as layer 105, include application of a ReLU activation function.

In some embodiments, each convolutional layer is followed by a pooling layer 106, where the output size is (W−Kₛ+1)/S. The pooling layer 106 downsamples its inputs. In the embodiment shown in FIG. 1, the pooling layer 106 employs average pooling to downsample its input. In other embodiments, max pooling is used. In the embodiment of FIG. 1, the output of the pooling layer has size 113×3.

In the embodiment of FIG. 1, a second convolutional layer 107 receives the output of the pooling layer 106, and performs another convolution operation on the data. In this embodiment, the output of the second convolutional layer 107 has size (111,3) and includes ReLU activation. In this embodiment, a second pooling layer 108, which uses average pooling, takes the output of the second convolutional layer 107 and has output of (55,3). In this embodiment, a final, third convolutional layer 109 performs a third convolution operation and outputs a matrix of size (55,3). In some embodiments, including a convolutional layer as the final layer in the feature extraction block is helpful to enable a Grad-CAM analysis, as discussed further below.

As discussed below, in some embodiments, the weights of the convolutional and pooling layers are trained by passing training input spectra through each of these layers during a forward training pass. Then, a loss function is evaluated, and weights are updated during the backward propagation of error. In some embodiments, some of the hyperparameters in the convolutional neural network architecture were tuned using KerasTuner, including the number of filters in the convolutional layers, number of neurons in the dense layer after flattening, and the learning rate.

The output of the feature extraction block (i.e., the convolution/pooling process) is a matrix. The components of the matrix are the derived set of values containing the most relevant information contained in the unput spectrum, now in the form of a lower-dimensional vector. In some embodiments, this output matrix is converted to a vector in a flattening operation and then fed into the classification block 110 for determination of the sample component(s). In some embodiments, the classification block comprises a fully connected dense neural network. In some embodiments, the classification block comprises, as instructions on the computer-readable medium executable by the processor, at least one dense layer 111 and an output layer 112, for classifying the set of derived values from the feature extraction block as indicative of one or more materials in the sample. In some embodiments, each dense layer applies weights and biases and nonlinear ReLU activation. Other embodiments have other numbers of layers in the classification block: some embodiments include a plurality of dense layers and a softmax layer as the output layer.

The terms “classification block” are used for ease of understanding some of the functions of the technology, and are not intended to limit the structure or grouping of software code or modules used in embodiments of the present technology.

In the embodiment of FIG. 1, a first dense layer 111 has output of size (159,1). In the embodiment shown, this output is fed to each neuron of a second dense layer 113, which has output size (275,1). The output of the second dense layer 113 is provided to the output layer 112, which is a dense softmax layer. The second dense layer 113 is sometimes referred to as a hidden layer. In some embodiments, each layer of the neural network has a decreasing number of neurons, where the final output has a neuron for each possible mixture of the considered compounds. Each neuron in the output layer provides a raw score for each mixture class, comprising one or more materials in the sample. In the embodiment of FIG. 1, there are 255 neurons corresponding to the 255 mixtures possible from 8 total components. As discussed below, 8 mixture components were used to train the model, which, presents an 8-label classification problem. Using a label powerset method, the 8-label classification problem is converted to a 2⁸−1=255 class classification problem. In some embodiments, therefore, the number of neurons in the output layer 112 is equal to the number of possible mixtures that the system is trained to detect. In other embodiments, other numbers of components are used, which results in different numbers of possible mixtures and corresponding numbers of neurons in the output layer.

In the embodiment of FIG. 1, each neuron of the output layer provides a raw score for each mixture class, and these scores are converted to estimated probabilities via softmax activation. The softmax scores are given by:

$$Softmax ( {𝓏}_{i} ) = \frac{{e}^{{𝓏}_{i}}}{{\sum }_{j = 1}^{k} {e}^{{𝓏}_{j}}}$$

where zᵢ represents the raw score or logit for a specific class (unique gas mixture), i. It is also the input to the softmax layer in the neural network (the output layer). The numerator is the exponential of the raw score for class i. The denominator is the sum of the exponentials of raw scores for all k classes (unique combinations of mixture components). In some embodiments, the softmax score is compatible with cross-entropy loss, offers stability in terms of model training, and highly penalizes the deep neural network for incorrect classifications. In this manner, the system 101 measures the spectrum of a gas mixture of interest, and then outputs the estimated probabilities for the mixture speciation.

In some embodiments, the model comprising the feature extraction block and classification block was trained using spectra for mixtures containing one, two or three gaseous components. In the embodiment of FIG. 1, the model comprising the feature extraction block and classification block was trained using simulated mixture spectra. In other embodiments, experimentally generated spectra are used for model training.

In the embodiment of FIG. 1, to generate simulated mixture spectra, simulated spectra for the following eight pure components were used:
acetaldehyde CH₃CHO
acetonitrile CH₃CN
chloromethane CH₃Cl
methanol CH₃OH
ethanol C₂H₅OH
formic acid HCOOH
nitric acid HNO₃
formaldehyde H₂CO

The spectral absorbance of a gas mixture, Aₘᵢₓₜᵤᵣₑ, is given by:

$${A}_{mixture} = - ln ( \frac{I}{{I}_{0}} ) = {\sum }_{i} {ε}_{i} {c}_{i} l$$

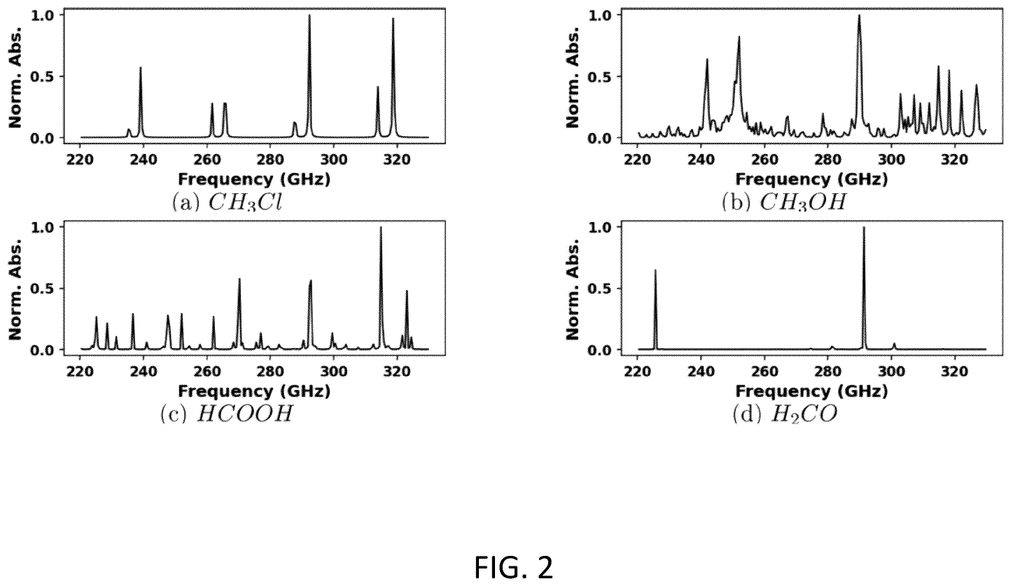

Simulations were carried out using the HAPI software and the spectroscopic parameters found in the HITRAN and JPL databases. The spectra were simulated at a frequency resolution of 0.016 cm−1, with a total pressure of 1 Torr, a temperature of 297 K, and a path length of 21.6 cm without dilution. Images of samples of the simulated spectra used in this embodiment, which correspond to pure CH₃Cl, CH₃OH, HCOOH, and H₂CO, are shown in FIGS. 2a-2d.

In this embodiment, mixture spectra were generated by the linear combination of the eight pure component reference spectra with randomly generated concentrations for each pure component. The simulated mixture spectra generated by a linear combination of the pure compound reference spectra assume collisional line broadening that is independent of mixture composition (i.e., all collisions result in broadening that is the same as a self-broadening collision).

For simulated spectra in this embodiment, the composition of the mixture is generated randomly by the selection of component concentrations for the linear combination from a uniform distribution on the interval [0, 1], while obeying two constraints. First, the maximum absorbance for each species in the mixture must be above 0.01. For kHz-rate experiments, minimum detectivity typically occurs for an absorbance of around 0.001. Hence, the requirement of a maximum absorbance of 0.01 for each species corresponds to a maximum signal-to-noise ratio (SNR) of 10 for the detection of each species at its peak value of absorbance within the 220-330 GHz frequency range. As a second constraint, so that each component absorbance sufficiently stands out compared to the total absorbance, the ratio of the maximum absorbance for each species to the maximum absorbance for the mixture should be 0.01 or greater. In this embodiment, these thresholds help ensure that the deep learning model will not learn from a spectrum which (a) is very weak, (b) contains practically undetectable components, and/or (c) has weaker absorbers whose fingerprint is overwhelmed by strong absorbers.

In this embodiment, then, the spectra generation process is as follows:
Step 1: A set of random concentration values for 8 components and the diluent are generated such that their sum equals 1.
Step 2: Using reference spectra at 1 Torr for the 8 pure compounds, a mixture spectrum is calculated via linear combination.
Step 3: The mixture spectrum is checked for the two constraints on absolute and relative absorbance.
Step 4: If the spectrum passes the two constraints, it is retained; otherwise, steps 1-3 are repeated.
Step 5: Once the desired number of mixture spectra are generated, 90 spectra from each of the 255 mixture classes are randomly sampled, to generate an unbiased data set with each mixture type equally represented.
Step 6: These spectra are split into a training and a validation set.

In this embodiment, for the unique 255 mixture types, 22950 unique simulated spectra were generated for the development of the system of FIG. 1 (90 spectra per mixture type). Approximately 97% of the simulated spectra were multicomponent, while the remaining 3% of the training spectra were for pure components diluted in air, such that the trained network would also accurately predict those single absorber mixtures. In this embodiment, the spectra were split into 60%-40% training and validation sets, yielding 13770 training spectra (54 spectra per mixture type) and 9180 validation spectra (36 spectra per mixture type). Thus, the matrices containing the training and validation spectra have shapes of 13770×229 and 9180×229, respectively, and spectra having a variety of concentrations for each component are available for model training.

In this embodiment, training was stopped after 37 epochs with accuracies on training and validation at 97.1% for both. The input and output layers of the network are trained using simulated spectral data sets and their corresponding multiclass integer label indices. During training, the Adam optimizer is used to update the weights and biases in the dense layers of the neural network based on the sparse categorical cross-entropy loss function with a batch size of 32.

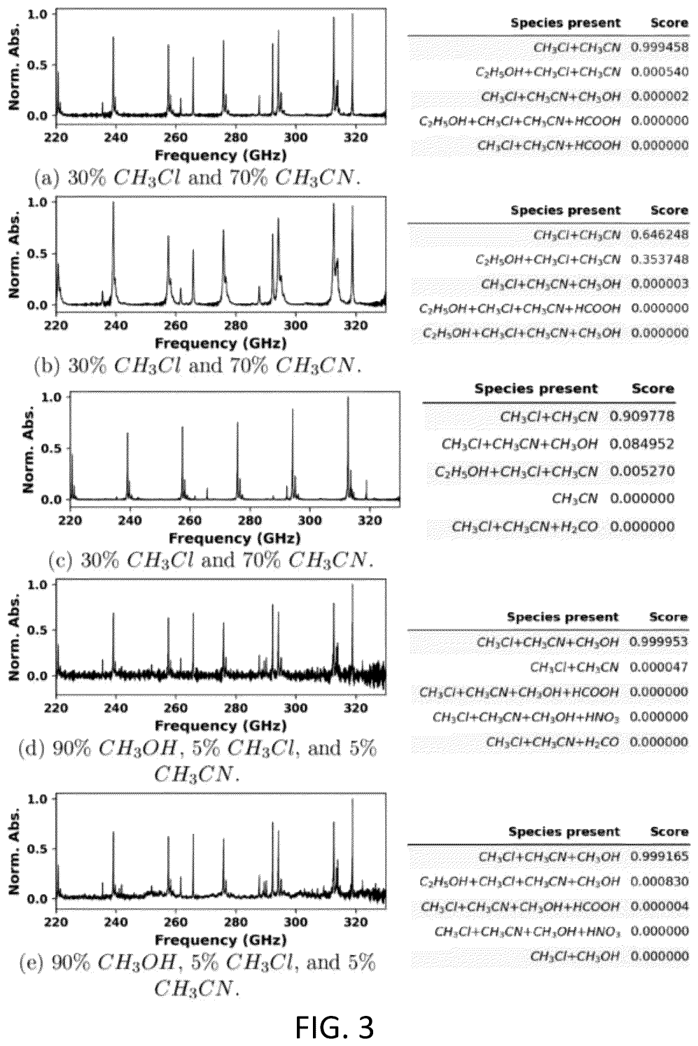

FIG. 3 shows classification performance of the embodiment of FIG. 1 on five experimental mixture spectra. The conditions for these experimental spectra were as follows:
a) 30% CH₃Cl and 70% CH₃CN diluted in 50% N₂
b) 30% CH₃Cl and 70% CH₃CN
c) 30% CH₃Cl and 70% CH₃CN diluted in 90% N₂
d) 90% CH₃OH, 5% CH₃CN, and 5% CH₃Cl
e) 90% CH₃OH, 5% CH₃CN, and 5% CH₃Cl

The measured spectra and the corresponding softmax scores from the system are shown. The system outputs 255 softmax scores corresponding to the probabilities associated with the respective spectra belonging to any one of the mixture classes. In FIG. 3, the top 5 softmax scores are given. Three measurements of 30% CH₃Cl-70% CH₃CN are illustrated and all correctly classified. The first (FIG. 3a) shows only high probability for the correct classification (CH₃Cl and CH₃CN), but the second two (FIGS. 3b and c) show marginal probabilities (35% and 8%) that the mixture could include C₂H₅OH in addition to CH₃Cl and CH₃CN. FIGS. 3d and e demonstrate two experimental spectra for a 3-component mixture (90% CH₃OH-30% CH₃Cl-70% CH₃CN) with different levels of noise. The model yields 99.9% probability for the correct classification in both cases and illustrates that the model can deal with variations in the noise floor.

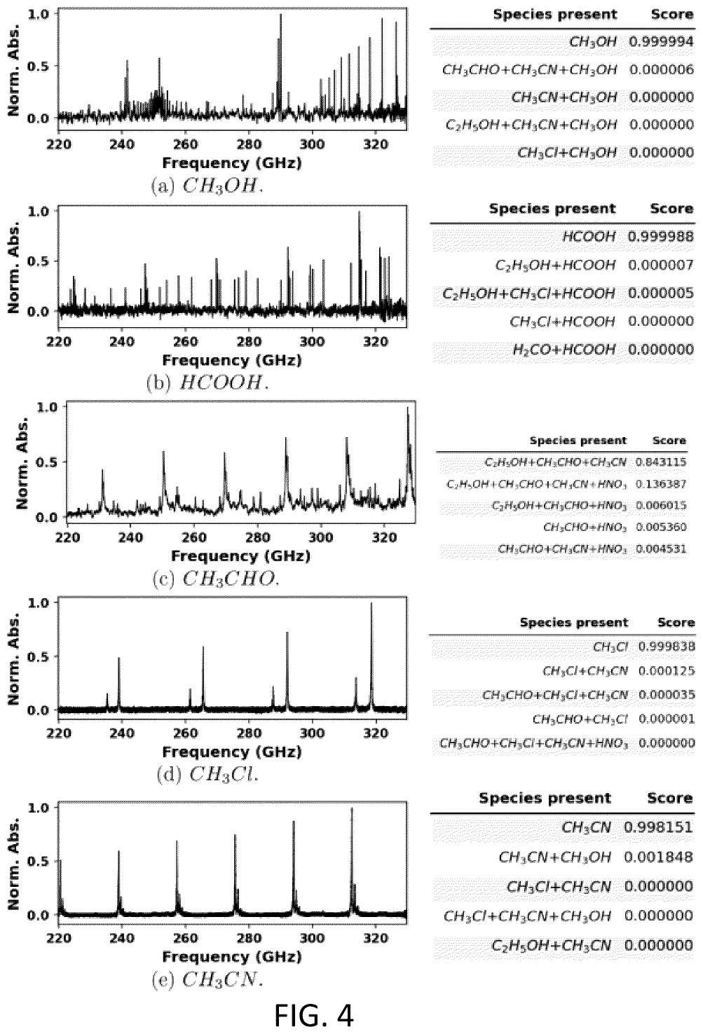

FIG. 4 shows measured pure spectra, for five single-component species: CH₃OH, HCOOH, CH₃CHO, CH₃Cl, and CH₃CN (which was acquired at 0.5 Torr). With the exception of acetaldehyde (CH₃CHO), the model produces the correct classification with over 99% probability. The acetaldehyde spectrum is misclassified as a three-component mixture of acetaldehyde, ethanol, and acetonitrile, with a reported probability of approximately 84%. For this misclassified spectrum, the model predicts acetaldehyde in all of the top five classification probabilities but predicts false positives. In this case, the model is misconstruing some of the absorption features within the acetaldehyde spectrum as arising from other species, which have similar and overlapping absorption features in this frequency range.

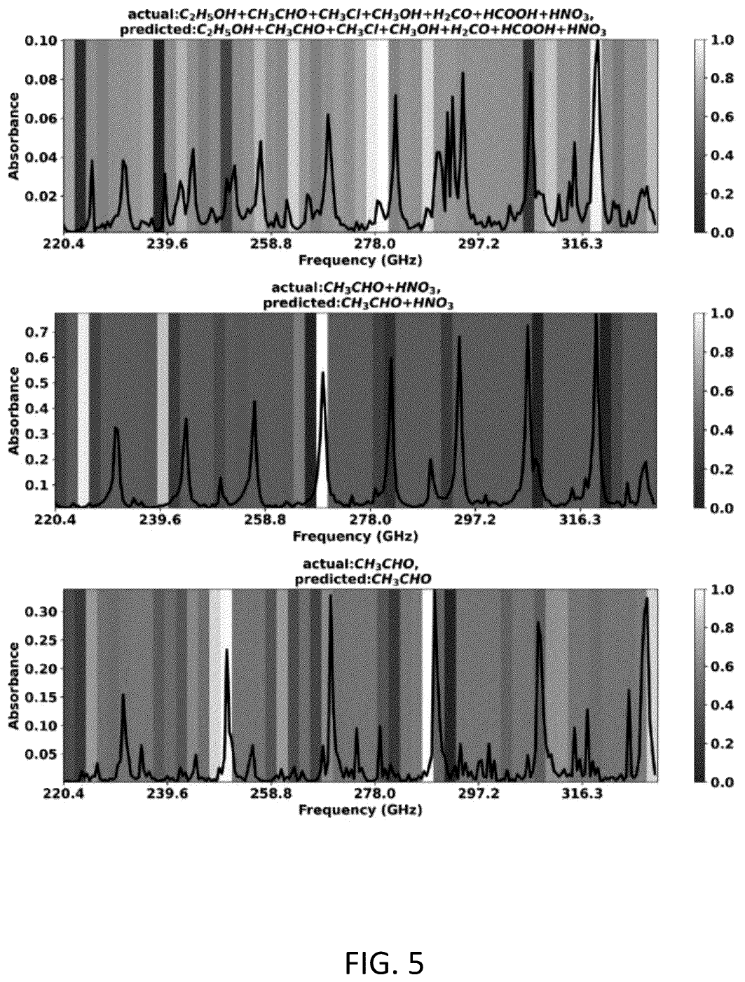

In this embodiment, gradient-weighted class activation mapping (Grad-CAM) has been employed to interpret the operations of the convolutional neural network employed in the system of FIG. 1. The last convolutional layer 109 within the model produces a set of extracted features which originate from the input features. Grad-CAM gives a visualization of the importance of each extracted feature using a heat map that is superimposed on the original raw spectrum, where the intensity (color) of the output indicates the weight associated with each extracted feature. FIG. 5 shows examples of such color maps, derived from the model of FIG. 1. The color scale indicates whether a feature has a positive or negative contribution to the model class prediction. Higher weights associated with extracted features indicate a positive contribution (bright colors) toward the identification of the positive class, and lower weights indicate a negative contribution (dark colors) to the final classification. The class activation maps contain useful information for sensor design, since critical localized spectral peaks within a spectrum for classification decision-making can be visualized, thus distinguishing mixture-discriminating regions in each spectrum.

Three class activation maps (CAM) are illustrated in FIG. 5. The example 7-component CAM (top graph) demonstrates that a significant portion of the spectrum is used to identify the mixture; i.e., much of the spectrum has colors that tend toward higher Grad-CAM weights (i.e., lighter colors). For mixtures containing fewer components, smaller portions of the spectra contribute positively to the mixture classification. In the cases of 2-and 1-component mixtures a smaller number of important features are identified by the model as providing the class-discriminating fingerprint.

In another embodiment, a system for classifying materials is provided for processing spectra in the IR frequency range (instead of the range of 220 to 330 GHz utilized by the system in FIG. 1). In this alternative embodiment, a wavenumber range of 400-4000 cm⁻¹ (wavelength of 2.5-25 micrometers) at 1 cm⁻¹ resolution is used to generate spectrum values for input into the model, which results in 3601 absorbance values (i.e., spectrum values). Other embodiments use alternative ranges, for example: 500-2000 cm⁻¹ (5-20 μm, n=1501); 1000-1500 cm⁻¹ (6.67-10 μm, n=501); 1250-1500 cm⁻¹ (6.67-8 μm, n=251); 1000-2000 cm⁻¹ (5-10 μm, n=1001); and 2000-4000 cm⁻¹ (2.5-5 μm, n=2001).

In this embodiment, the structure of the feature extraction block and classification blocks are similar to the embodiment of FIG. 1: a first convolutional layer followed by a first pooling layer, a second convolutional layer followed by a second pooling layer, and a third convolutional layer in the feature extraction block, and a first dense layer, a second dense layer, and an output layer in the classification block. Each convolution converts the input vector to a new vector whose size is given by (1/S)(W−Kₛ+2P)+1, where W, Kₛ, P, and S are the size of the input, kernel, padding, and the stride, respectively. In this embodiment, the output of the first convolutional layer with three filters is given by (1/1)(3601−3+2×0)+1=3599×3, where the sizes of the input vector, kernel, and stride are 3601, 3, and 1, respectively, and valid (zero) padding was used. In this embodiment, for a stride size of two and a pooling kernel size of two, the total output shape of the first pooling layer is 1799×3 for the range 400-4000 cm⁻¹. The output of each pooling layer is (W−Kₛ+1)/S.

In this embodiment, the matrix output of the feature extraction block is flattened to a vector and sent to the classification block, a fully connected dense neural network, for classification. In this embodiment, each dense layer applies learned weights and biases followed by nonlinear ReLU activation. In this embodiment, the Adam optimizer was implemented to update weights and biases using a sparse categorical cross-entropy loss function. A batch size of 32 was used for training, and the network was trained for 40 epochs. With the application of a softmax layer with 175 neurons, the final output from the neural network is a vector containing 175 softmax scores, each corresponding to a gas mixture. The model of this embodiment was trained using simulated spectra for 120 unique 3-component mixtures, 45 2-component mixtures, and 10 1-component mixtures (where ten gas species were used to generate the spectra) for a total of 175 unique gas mixtures.

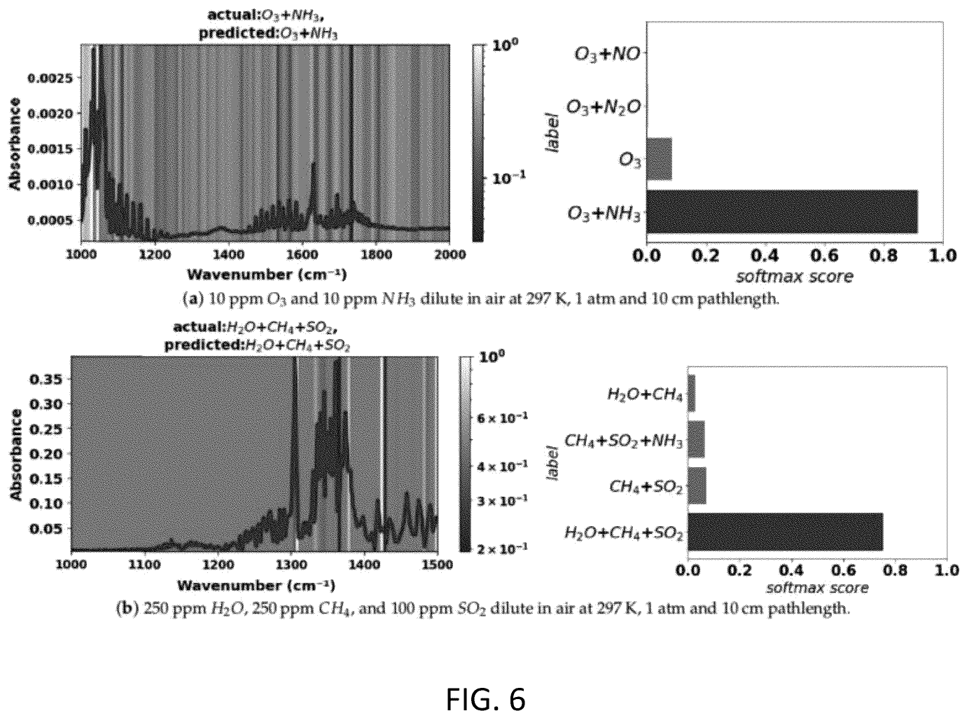

FIG. 6 shows two spectra, Grad-CAM heat maps, and the corresponding model mixture prediction softmax scores for the model of this embodiment against experimental data. Two synthetic experimental spectra were constructed from experiments for pure compounds reported in the National Institute of Standards and Technology molecular spectroscopy database. The experimental spectra were formed by concentration-weighted linear mixing of pure experimental spectra from that database. One mixture of spectra comprises ozone and ammonia in the 1000-2000 cm⁻¹ range. The other mixture of spectra comprises water vapor, methane, and sulfur dioxide in the 1000-1500 cm⁻¹ range. The model predicts the correct mixtures based on these spectra with a high degree of confidence, as shown by the softmax scores.

For the ozone-ammonia mixture, the model prioritizes absorption lines near 1070 cm⁻¹ for classification of ozone. Lines from 1450 to 1750 cm⁻¹, as well as some lines from 1150 to 1200 cm⁻¹, allow the model to correctly classify ammonia. For the water vapor-methane-sulfur dioxide mixture in the 1000-1500 cm⁻¹ range, the model prioritizes a strong feature around 1300 cm⁻¹ for the classification of methane. Sulfur dioxide is classified by the model using features around 1330 and 1380 cm⁻¹. And the model prioritizes features from 1400 to 1500 cm⁻¹ for the classification of water vapor.

In some embodiments, the size of the input absorbance vector varies based on the frequency range; thus, across different embodiments, the number of convolution and pooling blocks is varied to ensure that there are a sufficient number of neurons in the final layer to perform classification. The architecture of models according to the present technology is flexible to accommodate user-defined and variable input frequency bands (variable input vector lengths) requiring variable degrees of downsampling in the feature extraction block to extract a low-dimensional representation of the input spectrum.

Other embodiments of the present technology provide methods for classifying material samples. In some embodiments, the method comprises receiving a set of spectrum values extracted from a spectrum of a sample; reducing, in a feature extraction block comprising at least one convolutional layer and at least one pooling layer, the set of spectrum values to a set of derived values each indicative of a spectral feature of the spectrum of the sample; and classifying, in a classification block comprising at least one dense layer and an output layer, the set of derived values as indicative of one or more materials in the sample.

In some embodiments, the method further comprises combining reference spectra of one or more gaseous components to form a set of input spectra; converting each input spectrum to set of input values; passing a selected set of the input spectra, each as a set of input values, through the at least one convolutional layer, the at least one pooling layer, the at least one dense layer, and the output layer to obtain a set of training results corresponding to the set of input spectra; and updating, for each training result, one or more weights and/or one or more biases in at least one of the at least one convolutional layer, the at least one pooling layer, the at least one dense layer, and the output layer based on each training result. In some embodiments, the method further comprises the step of evaluating a loss function based on the training result to determine which of the one or more weights and/or one or more biases to update in the updating step. In some embodiments, a sparse categorical cross-entropy loss function with a batch size of 32 is used.

Some embodiments of the present technology include non-transitory computer-readable storage media and/or devices having stored thereon instructions that when executed by one or more processors perform the methods and processes described herein. The processor may include, for example, a processing unit and/or programmable circuitry. The storage device may include a machine readable storage device including any type of tangible, non-transitory storage device, for example, any type of disk including floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, magnetic or optical cards, or any type of storage devices suitable for storing electronic instructions.

Embodiments of the present technology include systems and methods for classifying material samples as described herein using one or more convolutional neural networks, as well as systems and methods for training convolutional neural networks or other machine learning models/systems for performing such classification.

As used herein, the terms “logic,” “block,” and/or “module” may refer to an app, software, firmware and/or circuitry configured to perform any of the aforementioned operations. Software may be embodied as a software package, code, instructions, instruction sets and/or data recorded on non-transitory computer-readable storage media. Firmware may be embodied as code, instructions or instruction sets, and/or data that are hard-coded (e.g., nonvolatile) in memory devices.

“Circuitry”, as used herein, may include, for example, singly or in any combi-nation, hardwired circuitry, programmable circuitry such as computer processors comprising one or more individual instruction processing cores, state machine circuitry, and/or firmware that stores instructions executed by programmable circuitry. The logic and/or module may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc.

Although the technology has been described and illustrated with respect to exemplary embodiments thereof, it should be understood by those skilled in the art that the foregoing and various other changes, omissions and additions may be made therein and thereto, without departing from the spirit and scope of the present invention.

What is claimed is:

1. A system for classifying material samples, comprising:
a processor;
a computer-readable storage medium comprising instructions executable by the processor for:
receiving spectrum signal data related to a spectrum of a sample;
converting the signal data into a set of spectrum values;
reducing, in a feature extraction block comprising at least one convolutional layer and at least one pooling layer, the set of spectrum values to a set of derived values each indicative of a spectral feature of the spectrum of the sample; and
classifying, in a classification block comprising at least one dense layer and an output layer, the set of derived values as indicative of one or more materials in the sample.

2. The system of claim 1, wherein the feature extraction block further comprises a first convolutional layer, a second convolutional layer, and a first pooling layer between the first and second convolutional layers.

3. The system of claim 1, wherein the feature extraction block further comprises a first convolutional layer followed by a first pooling layer, a second convolutional layer followed by a second pooling layer, and a third convolutional layer.

4. The system of claim 1, wherein the classification block further comprises a fully connected dense neural network comprising a plurality of dense layers and a softmax layer as the output layer.

5. The system of claim 1, wherein the instructions for reducing further comprises applying three filters with a kernel size of three at the at least one convolutional layer.

6. The system of claim 4, wherein the classification block further comprises a hidden layer between the plurality of dense layers and the output layer.

7. The system of claim 1, wherein the feature extraction block and classification block were trained using spectra for mixtures containing one, two or three gaseous components.

8. A method for classifying material samples, comprising the steps of:
receiving a set of spectrum values extracted from a spectrum of a sample;
reducing, in a feature extraction block comprising at least one convolutional layer and at least one pooling layer, the set of spectrum values to a set of derived values each indicative of a spectral feature of the spectrum of the sample; and
classifying, in a classification block comprising at least one dense layer and an output layer, the set of derived values as indicative of one or more materials in the sample.

9. The method of claim 8, wherein the feature extraction block further comprises a first convolutional layer, a second convolutional layer, and a first pooling layer between the first and second convolutional layers.

10. The method of claim 8, wherein the feature extraction block further comprises a first convolutional layer followed by a first pooling layer, a second convolutional layer followed by a second pooling layer, and a third convolutional layer.

11. The method of claim 8, wherein the classification block further comprises a fully connected dense neural network comprising a plurality of dense layers and a softmax layer as the output layer.

12. The method of claim 8, wherein the instructions for reducing further comprises instructions for applying three filters with a kernel size of three at the at least one convolutional layer.

13. The method of claim 11, wherein the classification block further comprises a hidden layer between the plurality of dense layers and the output layer.

14. The method of claim 11, further comprising the steps of:
combining reference spectra of one or more gaseous components to form a set of input spectra;
converting each input spectrum to set of input values;
passing a selected set of the input spectra, each as a set of input values, through the at least one convolutional layer, the at least one pooling layer, the at least one dense layer, and the output layer to obtain a set of training results corresponding to the set of input spectra; and
updating, for each training result, one or more weights and/or one or more biases in at least one of the at least one convolutional layer, the at least one pooling layer, the at least one dense layer, and the output layer based on each training result.

15. The method of claim 14, further comprising the step of evaluating a loss function based on the training result to determine which of the one or more weights and/or one or more biases to update in the updating step.

16. A non-transitory computer readable storage medium comprising instructions executable by a processor for:
converting spectrum signal data related to a spectrum of a sample into a set of spectrum values;
reducing, in a feature extraction block comprising at least one convolutional layer and at least one pooling layer, the set of spectrum values to a set of derived values each indicative of a spectral feature of the spectrum of the sample; and
classifying, in a classification block comprising at least one dense layer and an output layer, the set of derived values as indicative of one or more materials in the sample.

17. The storage medium of claim 16, wherein the instructions for reducing further comprises instructions for applying three filters with a kernel size of three at the at least one convolutional layer.

18. The storage medium of claim 16, further comprising instructions for:
combining reference spectra of one or more gaseous components to form a set of input spectra;
converting each input spectrum to set of input values;
passing a selected set of the input spectra, each as a set of input values, through the at least one convolutional layer, the at least one pooling layer, the at least one dense layer, and the output layer to obtain a set of training results corresponding to the set of input spectra;
updating, for each training result, one or more weights and/or one or more biases in at least one of the at least one convolutional layer, the at least one pooling layer, the at least one dense layer, and the output layer based on each training result.

19. The storage medium of claim 16, further comprising instructions for evaluating a loss function based on the training result to determine which of the one or more weights and/or one or more biases to update.

20. The storage medium of claim 16, wherein the feature extraction block further comprises a first convolutional layer followed by a first pooling layer, a second convolutional layer followed by a second pooling layer, and a third convolutional layer.
